(19) United States
(12) Patent Application Publication
(10) Pub. No.: US 2022/0081483 A1
(43) Pub. Date: Mar. 17, 2022

(54) METHODS AND COMPOSITIONS FOR REDUCING NUMBERS OR ELIMINATING HIV-INFECTED CELLS

(71) Applicant: The Wistar Institute of Anatomy and Biology, Philadelphia, PA (US)

(72) Inventors: Mohamed Abdel-Mohsen, Wynnewood, PA (US); Florent Colomb, Philadelphia, PA (US); Bertoni Leila Giron, Philadelphia, PA (US)

(21) Appl. No.: 17/295,084

(22) PCT Filed: Nov. 19, 2019
(86) PCT No.: PCT/US2019/062207

§ 371 Date: May 19, 2021

Related U.S. Application Data
(60) Provisional application No. 62/770,134, filed on Nov. 20, 2018.

(51) Int. Cl. C07K 16/28; A61P 31/18; G01N 33/569; G01N 33/533

(52) U.S. Cl. CPC C07K 16/2854; A61P 31/18; A61K 2039/505; G01N 33/533; G01N 33/56988

(57) ABSTRACT

A method of reducing or inhibiting migration of HIV-infected CD4+ T cells to tissues comprises contacting the infected cells with a ligand that prevents or inhibits the interaction between a T cell-surface fucosylated glycan and its glycan-binding protein or with a molecule that metabolically inhibits glycosylation or fucosylation in said cell. In vitro and in vivo methods are described. Also described is a diagnostic method employing labeled ligands that bind a fucosylated glycan and provide a signal detectable by non-invasive imaging.

### STATEMENT REGARDING FEDERALLY SPONSORED RESEARCH OR DEVELOPMENT

This invention was made with government support under Grant Nos. R21 AI129636 and P30 AI045008-19 awarded by the National Institutes of Health. The government has certain rights in this invention.

### BACKGROUND OF THE INVENTION

HIV infects 36.9 million people worldwide, 2.6 million of whom are children. Combination antiretroviral therapy (ART) has dramatically reduced morbidity and mortality for HIV-infected individuals in resource-rich countries with access to healthcare. However, more than 22 million people do not have access to ART, including 1.8 million children (UNAIDS report, 2015). Moreover, ART requires lifelong administration of at least three different medicines and does not eradicate HIV, which continues to cause immune activation, inflammation, ongoing damage to multiple organs systems, and reduction in life expectancy. These collective realities have prompted a renewed interest in developing more effective and accessible therapies that can lead to eradication, functional cure, or improved tolerance of lifelong infection.

The main barrier to HIV eradication is the ability of HIV to establish persistent infection in long-lived CD4+ T cells, which persist in the blood and at higher levels in tissues. These cells do not produce virus constitutively but can be induced by activation to produce infectious virus. Very little is known about the cell-surface characteristics of persistent HIV-infected cells during suppressive antiretroviral therapy (ART).

There remains a need in the art for effective methods and compositions for the treatment of HIV, and particularly the eradication of persistent infection.

### SUMMARY OF THE INVENTION

In one aspect, a method of reducing or inhibiting migration of HIV-infected CD4+ T cells from blood to tissues, such as uninfected tissues, is provided. This method involves contacting the infected cells with a ligand that prevents or inhibits the interaction between a T cell-surface fucosylated glycan and its glycan-binding protein or a molecule that metabolically inhibits glycosylation or fucosylation in said cell.

In another aspect, a method of reducing or inhibiting migration of HIV-infected CD4+ T cells from blood to uninfected tissues is provided. This method involves contacting the infected cells with a ligand that prevents or inhibits the interaction between a fucosylated Sialyl Lewis X (SLe^{x}) glycan and its Selectin binding protein.

In yet another aspect, a method of reducing HIV persistence comprises administering to a subject in need thereof a therapeutic agent that binds SLe^{x} or Selectin and inhibits their interaction, or that metabolically inhibits fucosylation or glycosylation. The administration of this therapeutic agent is performed substantially simultaneously with antiretroviral therapy.

In still a further aspect, a method of stimulating cellular immunity is provided. This method includes altering the glycans on the cell surface of cells infected with HIV, or manipulating host cell-surface glycan-lectin interactions. This method involves contacting the infected cells with a ligand that prevents or inhibits the interaction between a T cell-surface fucosylated glycan and its glycan-binding protein or a molecule that metabolically inhibits glycosylation or fucosylation in said cell.

In another aspect, a method is described for detecting HIV-infected cells in a subject comprising administering to said subject a ligand that binds to fucose-containing cell surface molecules, said ligand associated with a detectable label and performing a non-invasive imaging technique to detect said label. The increase in detection of the label over a control indicates the presence of HIV-infected cells. In one embodiment, the labeled ligand binds the molecule fucosylated SLe^{x}.

Yet another aspect involves a diagnostic reagent for non-invasive detection of HIV-infected cells or tissue comprising a ligand that binds to a fucose-containing cell surface molecule, associated with a fluorescent label. This reagent can be optionally associated with a second ligand, associated with a second detectable label, that binds to a surface protein or epitope on an HIV infected T cell, e.g., CD4.

In another embodiment, a diagnostic kit comprises one or more ligand that binds to a fucose-containing cell surface molecule, associated with a label that is detectable by non-invasive means, e.g., image; one or more second ligand that binds to a surface protein or epitope on an HIV infected T cell, a variety of different detectable labels for association with the ligands, the labels capable of being identified by non-invasive techniques.

Yet another aspect provides a bi-specific composition comprising a ligand that binds to a fucose-containing cell surface molecule, associated with a ligand that binds to a surface protein or epitope expressed on an HIV infected T cell, one or more of the ligands associated with a detectable label. A pharmaceutically acceptable carrier or excipient may be included in any of these compositions.

Another aspect provides a composition containing a mixture of different ligands, each ligand capable of binding to a fucosylated glycan, or the fucosylated glycan binding protein, or a protein in the fucosylation pathway, e.g., a fucosylation transferase (FUT), each ligand optionally associated with a detectable label. In another embodiment, the mixture includes a mixture of metabolic inhibitors of glycosylation or fucosylation. In another embodiment, the composition includes one or more ligands that bind to a surface protein or epitope expressed on an HIV infected T cell, optionally associated with a detectable label. A pharmaceutically acceptable carrier or excipient may be included in any of these compositions.

Still other aspects and advantages of these compositions and methods are described further in the following detailed description of the preferred embodiments thereof.

### BRIEF DESCRIPTION OF THE DRAWINGS

FIG. 1A shows that CD4+ T cell surface fucosylation associates with persistent HIV transcription in vitro. The glycomic signature of HIV+ transcriptionally-inactive CD4+ T cells clusters distinctly from that of uninfected-non-activated, uninfected-activated, and productively-infected cells. Cells were infected and sorted using the dual-reporter virus HIV latency model FIG. 1B is a heat map and dendrogram depicting normalized lectin-binding intensity. Heat colors show standardized Z-scores; red indicates higher binding and blue indicates lower binding. Arrows point to lectins specific for branched fucose and core fucose (such as UAEI and AOL).

FIG. 2A through 2F are graphs showing that CD4+ T cell surface fucosylation associates with persistent HIV transcription in vivo. CD4+ T cells from HIV+ ART-suppressed individuals were sorted based on their cell surface fucosylation and levels of HIV DNA (FIGS. 2B, 2D and 2F) and cell-associated HIV RNA (FIGS. 2A, 2C and 2E) were measured. Cells with low fucose (branched or core) exhibit lower levels of cell-associated HIV RNA, despite similar levels of HIV DNA (lower HIV transcription) in vivo. Mann-Whitney tests.

FIG. 3 shows that levels of CD4+ T cell surface fucosylation (core, left panel; and branched, right panel) correlate with cell-associated HIV RNA during suppressive ART. N=14 HIV+ ART+ individuals. Spearman's rank tests.

FIGS. 4A (a variance plot) and 4B (a heat map) show that the principal component analysis of the transcriptomic profiles of high-fucose and low-fucose CD4+ T cells from HIV+ ART+ individuals, shows a clear clustering.

FIG. 5 shows volcano plots highlighting the up- and down-regulated genes when high-fucose CD4+ T cells are compared to low-fucose CD4+ T cells. Analysis showing a differential expression of genes associated with carbohydrate metabolism, glycolysis, mTOR signaling, T-Cell trafficking, and ERK/MAPK signaling. The original plot was split into two for clarity.

FIGS. 6A-6C are plots showing that high-Fucose CD4+ T cells express high levels of fucosyltransferase 7 (FUT7); a fucosyltransferase that encodes Sialyl Lewis X. The three graphs show transcriptomic analysis of high-fucose CD4+ T cells vs. FUT7, GCNT1 and L-Selectin. The results suggest that high-Fucose CD4+ T cells harbor higher levels of the Sialyl Lewis X cell-surface marker. FIG. 6D is a schematic cartoon of SLe^{x}, in which circles are galactose, triangle is fucose; square is GlcNAc; and diamond is sialic acid.

FIG. 7 shows a Cybersort analysis of the transcriptomic data from high-fucose and low-fucose CD4+ T cells. High-fucose cells are mostly resting memory CD4+ T cells (population known to be enriched with HIV+ cells during suppressive therapy). H=High-fucose cells, and L=Low-fucose cells.

FIG. 8 provides a flowchart of the two fucosylation pathways.

### DETAILED DESCRIPTION

Methods and compositions are described herein that are useful for targeting cell-surface glycomic structures containing the monosaccharide fucose, including variations of Sialyl Lewis X and/or targeting cell-free and cell-surface ligands of fucose-containing glycans, including Selectins, to prevent trafficking of HIV-infected cells to tissues and treat HIV infection.

Given the central importance of lymphoid tissues as a source of the HIV reservoir during HIV infection, there is a tremendous interest in developing tools to manipulate HIV+ T cell trafficking between blood and tissues. The inventors have determined that HIV persistence in tissues can be impacted by preventing these HIV-infected transcriptionally-active memory CD4+ T cells from migrating from blood to tissues, through inhibitors of the interaction between cell surface glycans, e.g., SLe^{x}, and glycan-binding proteins, e.g., Selectins. Similarly, the inventors have determined that metabolic inhibitors of glycosylation and fucosylation can be used in accomplishing the same effect.

Without wishing to be bound by theory, the inventors hypothesize that a subset of HIV+ transcriptionally active memory CD4+ T cells express high levels of SLe^{x}, allowing them to traffic to tissues where ART penetration might be suboptimal, contributing to HIV persistence and chronic inflammation. SLe^{x} binding to the cell adhesion molecules, Selectins, allows leukocytes to leave the vascular tree and become recruited into tissues. In addition, T cell surface fucosylation is also known to be critical for T cell activation.

The inventors identified what is believed to be a first of its kind, cell-surface glycomic features of HIV+ transcriptionally cells during suppressive antiretroviral therapy (ART). The inventors have determined that CD4+ T cells with high cell-surface fucose express high levels of FUT7, low levels of L-Selectin, and high levels of GCNT1, suggesting that these cells with high fucose are memory CD4+ T cells. The HIV-infected cells can be identified, measured, and glycan signatures or profiles of such cells can be used to detect, visualize, kill, and/or manipulate the fate of these cells. Further, by changing the glycans on the cell surface of cells infected with HIV, and/or manipulating host cell-surface glycan-lectin interactions, one can stimulate cellular immunity (e.g., NK cells) against such viral infections and/or inhibit trafficking of these cells to HIV sanctuary sites, which can prevent viral seeding.

Further, the glycomic signature can be used to develop a non-invasive carbohydrate-based in vivo imaging probes for these cells. These probes can be used to reveal the dynamics of HIV infection and its reservoirs before and after HIV potential curative interventions against HIV-infected reservoirs.

### Definitions and Components Used in the Methods and Compositions

Unless defined otherwise in this specification, technical and scientific terms used herein have the same meaning as commonly understood by one of ordinary skill in the fields of biology, biotechnology and molecular biology and by reference to published texts, which provide one skilled in the art with a general guide to many of the terms used in the present application. The definitions herein are provided for clarity only and are not intended to limit the claimed invention.

The term “anti-retroviral therapy” or “ART” refers to treatment of individuals infected with human immunodeficiency virus (HIV) using anti-HIV drugs. The standard treatment consists of a combination of at least three drugs (often called “highly active antiretroviral therapy” or HAART) that suppress HIV replication. Antiretroviral medicines that are often used to treat HIV include: Nucleoside/nucleotide reverse transcriptase inhibitors, also called nucleoside analogs, such as abacavir, emtricitabine, and tenofovir. These medicines are often combined for best results. Nonnucleoside reverse transcriptase inhibitors (NNRTIs), such as efavirenz, etravirine, and nevirapine. Protease inhibitors (PIs), such as atazanavir, darunavir, and ritonavir. Entry inhibitors, such as enfuvirtide and maraviroc. Integrase inhibitors, such as dolutegravir and raltegravir. In one embodiment, ART is a combination of drugs efavirenz, tenofovir, and emtricitabine. Other combinations, without limitation, include: Dolutegravir, abacavir and lamivudine, Dolutegravir, tenofovir and emtricitabine, elvitegravir, cobicistat and tenofovir, and emtricitabine, raltegravir, tenofovir and emtricitabine, or ritonavir-boosted darunavir, tenofovir and emtricitabine.

The term “glycan” as used herein refers to a complex oligosaccharide composed of 10-15 monosaccharide residues. One or more glycan(s) can be covalently attached to a protein to form a glycoprotein(s), or to a lipid(s) to form a glycolipid(s). Most human proteins are modified by covalent attachment of glycans. Most glycans attached to proteins can be classified as N-glycans, attached through nitrogen of asparagine. or O-glycans, attached through oxygen of mainly serine or threonine. Glycans of interest for use in the glycomic signatures can include, without limitation, one or more of monosialylated structures, di-sialylated structures, trisialylated structures, tetrasialylated structures, agalactosylated structures, monogalactosylated structures, di-galactosylated structures, trigalactosylated structures, tetragalactosylated structures, low branched (monoantennary and diantennary) structures, high branched (triantennary and tetraantennary), structures with bisecting GlcNAc, antennary fucosylated structures, and core fucosylated structures.

Fucosylated carbohydrate structures are involved in a variety of biological and pathological processes in eukaryotic organisms including tissue development, angiogenesis, fertilization, cell adhesion, inflammation, and tumor metastasis. Fucosyltransferases (FuTs) are the enzymes that catalyze the inverting reaction in which a fucose residue is transferred from the donor guanosine-diphosphate fucose (GDP-Fuc) to the acceptor molecules including oligosaccharides, glycoproteins, and glycolipids (Ma, B et al, Fucosylation in prokaryotes and eukaryotes, Glycobiol., 16(12):158R-184R (December 2006)).

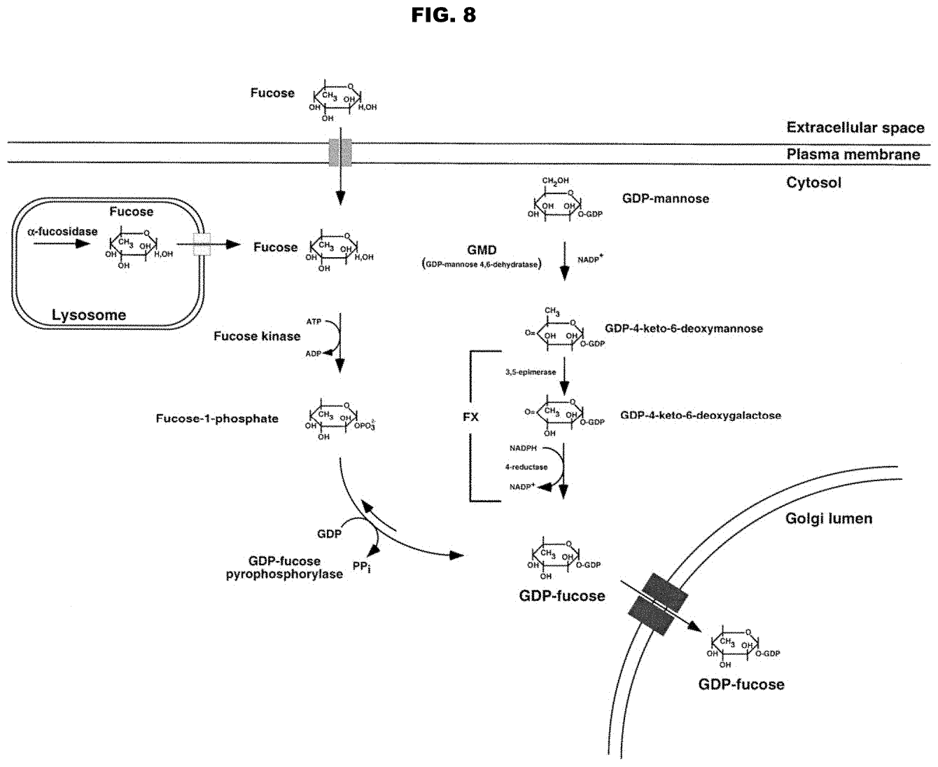

All fucosyltransferases utilize a nucleotide-activated form of fucose, GDP-fucose, as a fucose donor in the construction of fucosylated oligosaccharides. Two pathways have been described for synthesis of GDP-fucose in the cytosol of essentially all mammalian cells. The de novo pathway transforms GDP-mannose to GDP-fucose via three enzymatic reactions carried out by two proteins, GDP-mannose 4,6-dehydratase (GMD) and a second enzyme, GDP-keto-6-deoxymannose 3,5-epimerase, 4-reductase, also known as the FX protein. The salvage pathway synthesizes GDP-fucose from free fucose derived from extracellular or lysosomal sources. See FIG. 8 obtained from Becker, DJ., Fucose:biosynthesis and biological function in mammals, Glycobiol., 13(7):41R-53R (July 2003).

Sialyl-Lewis X (SLe^{x}) is a trisaccharide antigen of the formula C₃₁H₅₂N₂O₂₃ expressed on glycolipids and many cell-surface glycoproteins. In the blood the antigen is found on the surface of neutrophils; eosinophils; and monocytes. This carbohydrate antigen is accumulated in various human cancer tissues and secreted into the blood stream. The carbohydrate moiety can be further modified with fucose or sialic acid. The tetrasaccharide structure Siaα2,3Galβ1,4(Fucα1,3)GlcNAc constitutes the epitope of the carbohydrate antigen SLe^{x}. And is the minimal requirement for Selectin binding to their counter-receptors.

The term “glycome” as used herein refers to the set of all glycans in an organism/tissue/cell, or even of a single glycoprotein. In one embodiment, the glycome is the set of all glycans in the IgG of a human subject. In another embodiment, the glycome is the set of all glycans in the plasma of a human subject. In another embodiment, the glycome is the set of all glycans on the subject's cell-surface, either from all cells or from a selected cell type. In another embodiment, the glycome is the set of all gly cans in the subject's tissues. either from all tissues or from a selected tissue type. In another embodiment, the glycome is the subject's total exosome-bound glycome.

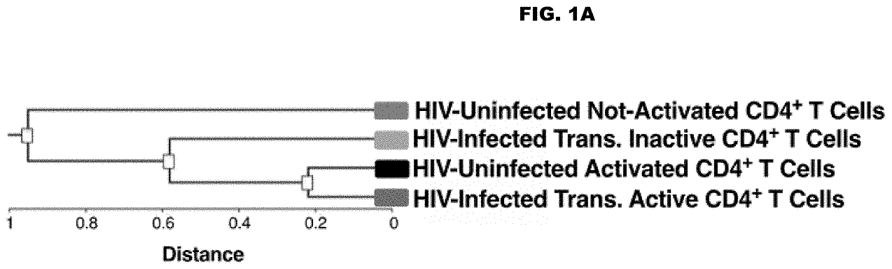

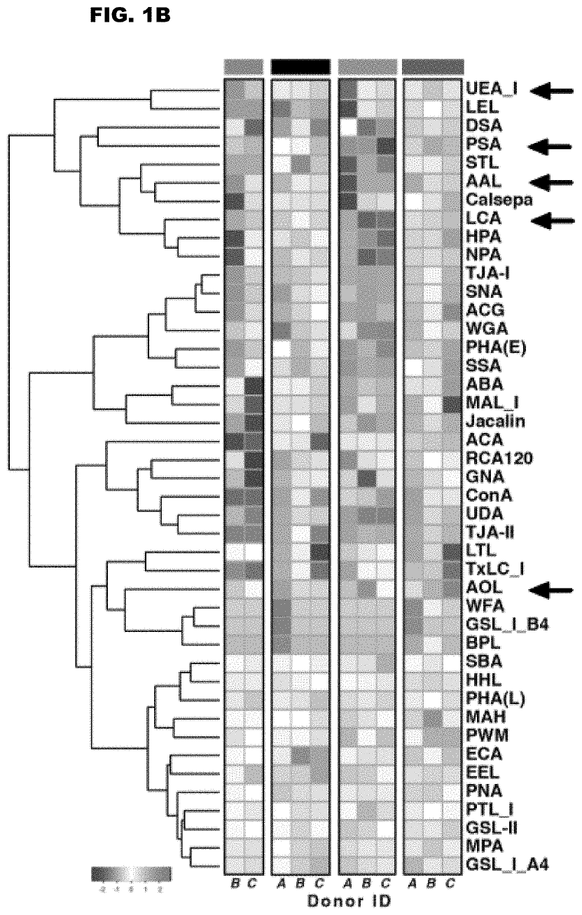

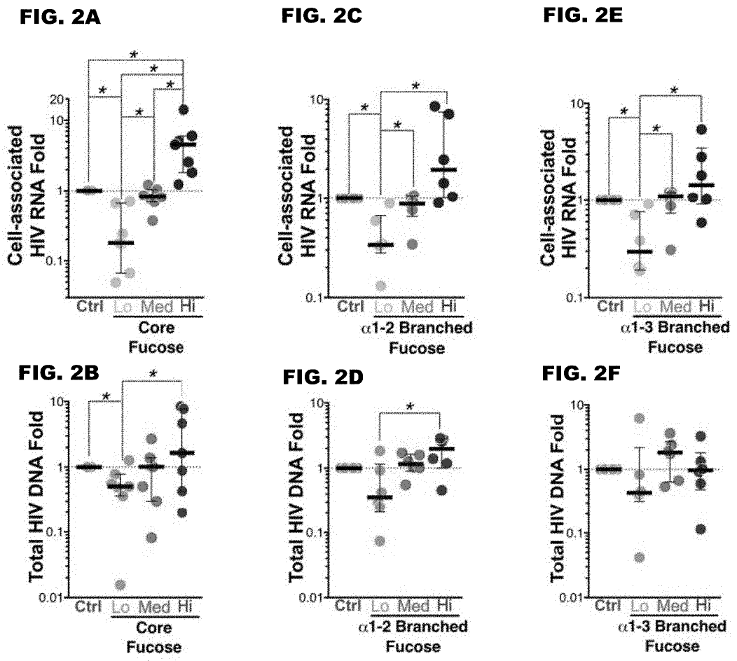

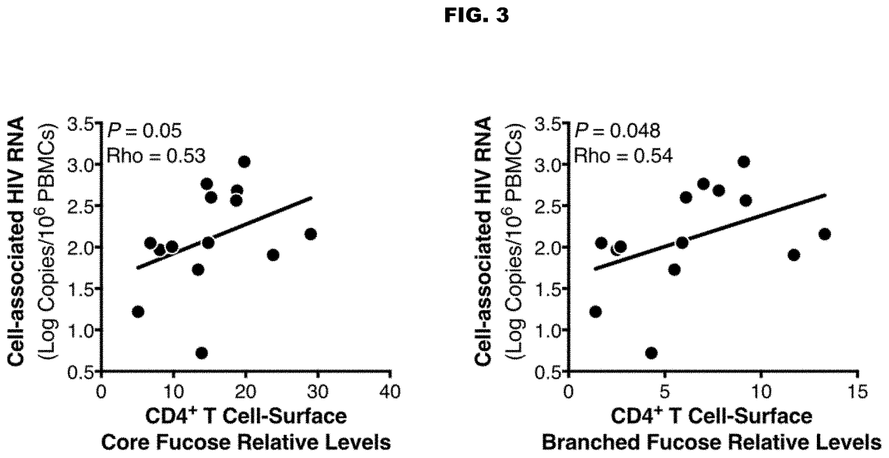

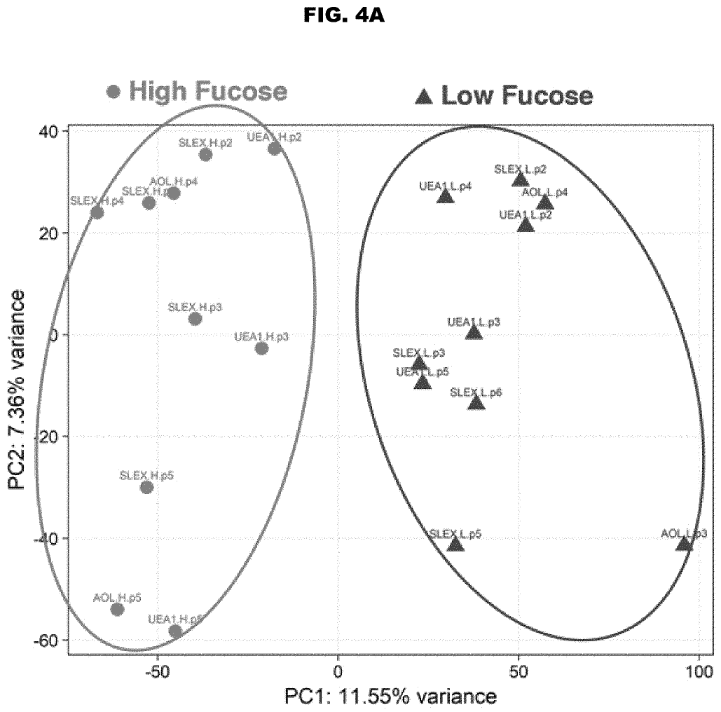

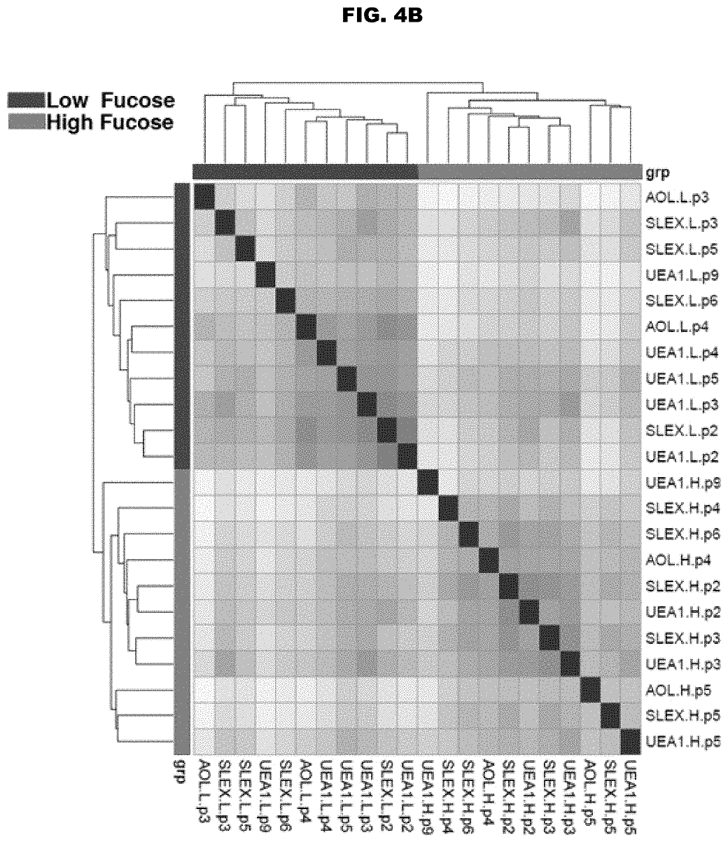

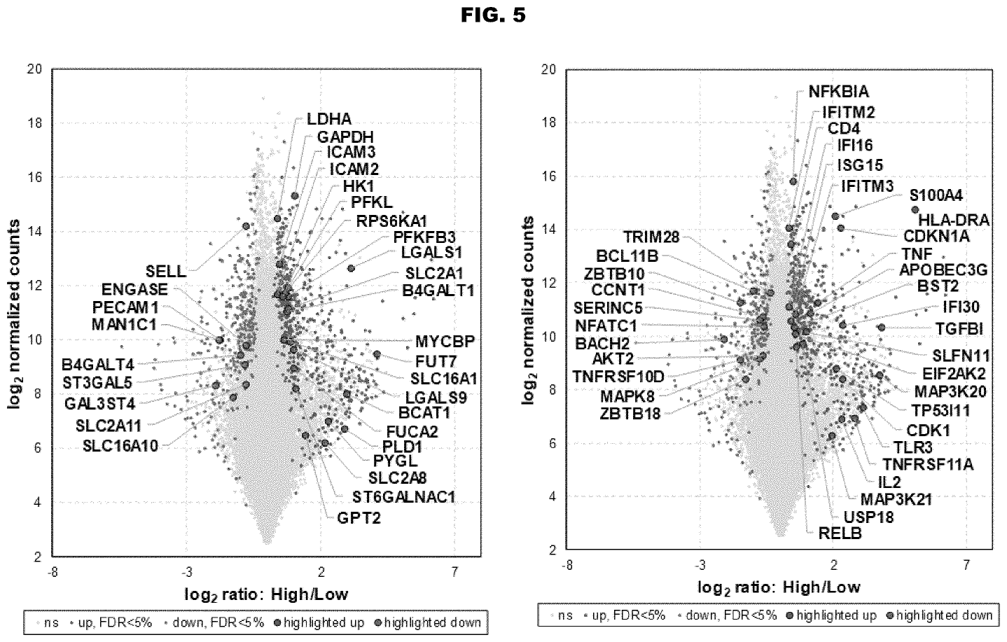

The term “glycomic signature” or “glycomic profile” as used herein refers to a pattern of one or more, or total glycosylated proteins or antibodies present in a biological sample of a human. A glycomic signature can be characteristic of a healthy state or a disease or disease state. In one embodiment, a glycomic signature characterized by hypo-fucosylation is a determined to be a characteristic of an HIV+ subject having a likelihood of HIV persistence. In one embodiment, a relevant glycomic signature of HIV persistence is high levels of fucosylation (see FIGS. 2A-2F showing core fucose and branched fucose) in T cells. Another embodiment of the glycomic signature of HIV persistence contains about 3000 genes. See FIG. 1B, specifically UEA_1, PSA, AAL, LCA, AOL. See, FIGS. 4A, left panel and 4B and all of the genes identified in FIG. 5.

The term “lectin” refers to a protein with a functional carbohydrate recognition domain which binds specific glycan structures, regarding both monomer composition and spatial arrangement.

The Selectins are a family of cell adhesion molecules (or CAMs) that bind to sugar moieties and so are considered to be a type of lectin. The three members of the selectin family bind carbohydrate structures through a Ca⁺⁺-dependent domain. They are transmembrane type I proteins with a short cytoplasmic tail and the long N-terminal portion containing the carbohydrate recognition domain protruding in the extracellular space. Selectins are found chiefly on the cell surface of white blood cells and mediate the binding of white blood cell to the endothelial cell, thus, are involved in host defense mechanisms, particularly the binding and rolling of the white blood cell to the endothelium. The Selectin family is comprised of three major isoforms: L-Selectin, E-Selectin and P-Selectin The three Selectins differ in their structure and pattern of cell type expression. L-Selectin is expressed on lymphocytes, monocytes and granulocytes, and is responsible for their homing through the binding of specific carbohydrate structures expressed by the high endothelial venules in lymph nodes. P-Selectin is expressed by platelets and endothelial cells and is stored in membranes of a granules of platelets and in Weibel-Palade bodies of endothelial cells. E-Selectin is constitutively expressed on the cell surface of venular endothelia of bone marrow and skin, while in other organs it is expressed upon stimulation by TNF-α, IL-1β, or LPS. The interaction between E-Selectin and its ligands expressed on the cell surface allows leukocyte rolling (Trinchera M, et al, Selectin Ligands Sialyl-Lewis a and Sialyl-Lewis X in Gastrointestinal Cancers, Biol (Basel), 6(1):16 (March 2017)).

As used herein, the term “ligand” refers to any naturally occurring or synthetic biological or chemical molecule which is used to bind specifically to a single identified target. As used herein, the targets are the glycan, e.g., SLe^{x}, the glycan binding protein, e.g., a Selectin, or a target protein in either of the fucose pathways of FIG. 8. The binding between the ligand and the target can be covalently or non-covalent, i.e., conjugated or by any known means considering the nature of the ligand and its respective target. A ligand may be selected independently from a peptide, a protein, an antibody or antibody fragment (e.g., an antigen binding portion of an antibody), an antibody mimetic, an affibody, a ribo-or deoxyribo-nucleic acid sequence, an aptamer, a lipid, a polysaccharide, a lectin, or a chimeric molecule formed of multiples of the same or different ligands or a small chemical molecule. Additional non-limiting examples of a ligand include a Fab, Fab′, F(ab′)2, Fv fragment, single-chain Fv (scFv), diabody (Dab), synbody, nanobodies, BiTEs, SMIPs, DARPins, DNLs, Duocalins, adnectins, fynomers, Kunitz Domains Albu-dabs, DARTs, DVD-IG, Covx-bodies, peptibodies, scFv-Igs, SVD-Igs, dAb-Igs, Knob-in-Holes, triomAbs, the like or combinations thereof. In some embodiments, a ligand is a recombinant or naturally occurring protein. In certain embodiments, a ligand is a monoclonal or polyclonal antibody, or fragment thereof. In certain embodiments, the ligand is a chemical compound that inhibits the target. In one embodiment, the ligand(s) of the constructs can also be directly labeled with one or more detectable labels, such as fluorophores (see labels discussed below) that can be measured by methods independent of the methods of measuring or detecting the polymer construct described otherwise herein.

By “inhibitors” as used herein is meant ligands that interfere with or inhibit the interaction between a glycan and its binding protein, such as a fucosylated glycan and its binding protein, e.g., fucosylated SLe^{x} and a Selectin.

By “Selectin-specific inhibitors” include those antibodies and small molecules known to inhibit one of the three members of the Selectin family. In one embodiment, the P-Selectin specific inhibitors include, without limitation, the monoclonal antibody (mAb) crizanlizumab (SEG101, SelG-1; Novartis) CAS 1690318-25-2; the mAb SelK-2 (Tetherex Pharma) that acts by targeting P-Selectin glycoprotein ligand-1 (PSGL-1); the mAb AbGn-168H (AbGenomics Intl Inc.); the small molecule GM-0111 (GlycoMira Therapeutics, Inc.) a semi-synthetic glycosaminoglycan (GAG) analogue based on chemically-modified hyaluronic acid; the small molecule pentosan polysulfate sodium (Vanguard Therapeutics Inc) CAS No. 140207-93-8; the synthetic peptide GsnP-6 (Ben-Gurion Univ); and the mAb inclacumab (Global Blood Therap., Inc) CAS #1256258-86-2. In another embodiment, the E-selectin specific inhibitors include, without limitation, the small molecule uproleselan sodium (GlycoMimetics Inc), formula C₆₀H₁₀₈N₃NaO₂; CAS No. 1914993-95-5; the small molecules GMI-1359, GMI-1687, and GMI-1757 (GlycoMimetics Inc.); and a polymer used to inhibit E-selectin (Ben-Gurion Univ). Still other antibodies or small molecules that bind or react with one of The Selectins so as to prevent or inhibit its interaction with SLe^{x} is considered to be useful in the methods and compositions described herein.

The term “pan-Selectin inhibitor” refers to a ligand that binds to or antagonizes binding with any of the three members of the family of Selectins, and is used in contrast to Selectin inhibitors that bind to or interfere with the binding of only one of the three Selectin members. Embodiments of pan-Selectin inhibitors include without limitation the small molecule rivipansel sodium or (GMI-1070; Pfizer Inc), CAS No. 1189037-60-2; and small molecules to inhibit P- and L-selectins by Tumorend LLC.

Other Selectin inhibitors include combinations or mixtures of any two or more Selectin-specific inhibitors, e.g., a combination of an E-selectin specific inhibitor with an L-selectin specific inhibitor with a P-selectin specific inhibitor. Still another embodiment of a Selectin inhibitor includes a construct which physically associates one or more of the mAB inhibitors or fragments thereof.

Glycan, fucosylated glycan or SLe^{x} inhibitors include without limitation antibodies to any of these glycans, such as anti-SLe^{x} antibodies such as 258-12767 by GeneTex, or the antibodies (Cat No. 0rb26220) by Biorbyt, or the dimeric antibodies (clone FH6) by BioLegend, or LS-0511275 from LifeSpan Biosciences, Inc. or the mAb for SLe^{x} (Chiba University) as well as other similar antibodies described in the publications incorporated by reference herein and known in the art.

By “metabolic inhibitor of glycosylation” is meant a molecule that blocks glycosylation by interfering with the metabolism of common precursors or intracellular transport activities. By “metabolic inhibitor of fucosylation” is meant a molecule that blocks fucosylation by interfering with the metabolism of common precursors or intracellular transport activities. Common precursors of fucosylation are shown in the pathways of FIG. 8. See e.g., Rillahan, C D et al., Global Metabolic Inhibitors of Sialyl-and Fucosyltransferases, Nat Chem. Biol., 2012 July; 8(7):661-668). Metabolic inhibitors of fucosylation include the SLe^{x} inhibitor small molecule SGN-2FF (SGD-2083 or2 fluoro-fucose; Seattle Genetics Inc.), which prevents the cells from making fucose-containing glycans (including SLex). See e.g, Clinical Trials.gov identifier NCT02952989. Other metabolic inhibitors of glycosylation in general or specifically fucosylation which can prevent the cells from making the fucose-containing glycans are also useful in these methods. Such inhibitors include those discussed in the art incorporated herein and known commercially or publicly.

By “receptor, epitope or protein expressed on HIV-infected cells” is meant one or more of the receptors, CD4, CCRS or CXCR4, among others (see the receptors listed in Clapham PR and McKnight, A., HIV-1 Receptors and Cell Tropism, Brit. Med Bull., 58(1):43-59 (September 2001) incorporated herein by reference, including mutations of these receptors. By “CD4” is meant a glycoprotein of approximately 60,000 molecular weight that is expressed primarily on the cell membrane of mature, thymus-derived (T) lymphocytes, and to a lesser extent on monocyte/macrophage lineage cells. The CD4 glycoprotein plays a role in mediating cellular immunity and also serves as the receptor for HIV. By “CCRS or CXCR4” are meant chemokine co-receptors also found on the CD4 T cell surface. Chemokine receptor 5 (CCRS), is used by macrophage-tropic (M-tropic) HIV to bind to a cell. About 90% of all HIV infections involve the M-tropic HIV strain. CXCR4, also called fusin, is a glycoprotein-linked chemokine receptor used by T-tropic HIV (ones that preferentially infect CD4 T-cells) to attach to the host cell.

As used herein, the term “detectable label” means a reagent, moiety or compound capable of providing a detectable signal, depending upon the assay format employed. A label may be associated one type of ligand only, so as to be distinguished from a mixture or combination of ligands. Such labels are capable, alone or in concert with other compositions or compounds, of providing a detectable signal. In one embodiment, useful labels include fluorescent compounds, fluorophores, radioactive compounds or elements. In one embodiment, a fluorescent detectable fluorochrome, e.g., fluorescein isothiocyanate (FITC), phycoerythrin (PE), allophycocyanin (APC), coriphosphine-O (CPO) or tandem dyes, PE-cyanin-5 or -7 (PC5 or PC7)), PE-Texas Red (ECD), PE-cyanin-5.5, rhodamine, PerCP, and Alexa dyes. Combinations of such labels, such as Texas Red and rhodamine, FITC+PE, FITC+PECyS and PE+PECy7, among others may be used depending upon assay method. Still other fluorescent protein labels include GFP, RFP and others. In another embodiment, the labels are desirably interactive to produce a detectable signal. The selection of a useful fluorescent protein label from among those commercially available is within the skill of the art.

Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength. Still other label systems that may be utilized in the described methods and constructs are detectable by other means, e.g., colored latex microparticles (Bangs Laboratories, Indiana) in which a dye is embedded may be used in place of enzymes to provide a visual signal indicative of the presence of the labeled ligand or construct in applicable assays The selection and/or generation of suitable labels for use in labeling the ligand and/or any component of the polymer construct is within the skill of the art, provided with this specification. Other components of the compositions and methods described herein can also be detectably labeled.

The detection of these labels is performed by known and preferably non-invasive imaging methods. By non-invasive detection methods is meant without limitation an in vivo imaging scanner, such as (but not limited to) magnetic resonance spectroscopy/imaging (MRS/MRI), positron emission tomography (PET) and other technology./imaging (MRS/MRI), positron emission tomography (PET) and other technology. However, depending upon the use of the labeled ligands, the detection or imaging method may be readily selected by one of skill in the diagnostic art.

As used herein, an “antibody or fragment” is a monoclonal antibody, a synthetic antibody, a recombinant antibody, a chimeric antibody, a humanized antibody, a human antibody, a CDR-grafted antibody, a multispecific binding construct that can bind two or more targets, a dual specific antibody, a bi-specific antibody or a multi-specific antibody, or an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, an Fc construct, a monovalent or bivalent construct from which domains non-essential to monoclonal antibody function have been removed, a single-chain molecule containing one V_{L}, one V_{H} antigen-binding domain, and one or two constant “effector” domains optionally connected by linker domains, a univalent antibody lacking a hinge region, a single domain antibody, a dual variable domain immunoglobulin (DVD-Ig) binding protein or a nanobody. Also included in this definition are antibody mimetics such as affibodies, i.e., a class of engineered affinity proteins, generally small (˜6.5 kDa) single domain proteins that can be isolated for high affinity and specificity to any given protein target. An antibody fragment or antigen binding fragment of an antibody refers to a portion of an antibody that binds specifically to a target and may include a Fab, Fab′, F(ab′)2, Fv fragment, single-chain Fv (scFv), scFv-Igs, and other fragments or portions of an antibody that can bind specifically to a target.

“Patient” or “subject” or “individual” as used herein means a mammalian animal, including a human, a veterinary or farm animal, a domestic animal or pet, and animals normally used for clinical research. In one embodiment, the subject of these methods and compositions is a human. In one embodiment, the subject is a human not diagnosed with HIV infection. In other embodiments, the subject is an HIV-infected subject who is symptomatic or non-symptomatic. In another embodiment, the subject is a human who has received, or is receiving, anti-retroviral therapy (ART). In another embodiment, the subject is a human who has discontinued ART. For diagnostic purposes, the subject may a healthy subject.

“Sample” as used herein means any biological fluid or suspension or tissue from a subject. In one embodiment suitable samples for use in the methods and with the diagnostic compositions or reagents described herein are samples or suspensions which require minimal invasion for testing, e.g., total plasma or isolated immunoglobulin G (IgG). Other samples include blood samples, including whole blood, peripheral blood, or serum, as well as cerebrospinal fluid, serous fluid, saliva or urine, vaginal or cervical secretions, and ascites fluids or peritoneal fluid or any tissues containing HIV reservoirs. In another embodiment, a suitable sample for use in the methods described herein includes peripheral blood, more specifically peripheral blood mononuclear cells. In another embodiment, the samples are concentrated by conventional means.

Control, control level, control signature or control profile as used herein refers to the source of the reference glycomic signature against which the tested subject's glycomic signature is analyzed, i.e., the levels of one or more selected glycans or total plasma, IgG, circulating, or HIV reservoir glycomes in a specified subject or in an average population of multiple subjects having a common stage of HIV infection or persistence. In one embodiment, the reference is biological samples selected from a reference healthy non-HIV-infected human subject or average population of such subjects. In another embodiment, the reference utilized is biological samples of a reference human subject or population of human subjects who are post-ART and demonstrate no HIV comorbidities and/or no HIV reservoirs. In another embodiment, the reference glycomic signature is a profile derived from the biological samples of the same human subject at a prior time, e.g., before or after ART, or before or after treatment with a therapeutic agent for manipulating the subject's glycomic signature. The control or reference standard, in various embodiments, is a mean, an average, a numerical mean or range of numerical means, a numerical pattern, a graphical pattern or a nucleic acid or gene expression profile derived from a control subject or a control population.

As used herein, the term “treatment” refers to any method used to alleviate, delay onset, reduce severity or incidence, or yield prophylaxis of one or more symptoms or aspects of an HIV infection. For the purposes of the present invention, treatment can be administered before, during, and/or after the onset of symptoms. In certain embodiments, treatment occurs after the HIV+ subject has received ART. In some embodiments, the term “treating” includes abrogating, substantially inhibiting, slowing, or reversing the progression of a condition, substantially ameliorating clinical or aesthetical symptoms of a condition, or substantially preventing the appearance of clinical or aesthetical symptoms of a condition, or decreasing the severity and/or frequency one or more symptoms resulting from the disease. More specifically treatment includes manipulating the level of the selected glycan to reduce HIV+ persistence, decrease the HIV+ reservoir, and/or reduce the severity, delay the onset, or prevent the development of a HIV comorbidity. In another embodiment, where the glycan is fucose, treatment involves administering compositions or therapeutic agents that operate to decrease the levels of fucose in the subject.

By “therapeutic agent” as used herein means any compositions that can be used to manipulate the host glycome, including modifying the levels of fucosylated glycans, e.g., SLe^{x} in the subject's total glycome to reduce the HIV reservoir and ameliorate HIV persistence. In one embodiment, the therapeutic agent is a selected glycan inhibitor in or associated with a suitable pharmaceutical carrier or excipient. In one embodiment, the therapeutic agent is a selected glycan binding protein inhibitor, such as a Selectin, in or associated with a suitable pharmaceutical carrier or excipient. In another aspect, the therapeutic agent is a metabolic inhibitor of glycosylation, fucosylation or of the glycan —glycan binding protein interaction. In still another embodiment, the therapeutic reagent is an inhibitor of the glycan or precursor in the glycan biosynthetic pathway or derivative thereof or a glycosylation inhibitor or deglycosylation enzyme, which can reduce the over-production of the selected glycan. Still other therapeutic reagents can include compounds or chemical moieties that can manipulate glycosyltransferase expression or fucosyltransferase expression or activity. Any of the active therapeutic reagents can be associated with known carriers or excipients, such as taught in the prior art.

The term “therapeutically effective amount” or “effective amount” refers to an amount agent that when administered alone or in combination with an additional therapeutic agent to a cell, tissue, or subject is effective to prevent or ameliorate HIV infection or HIV trafficking from the blood to a tissue, or amelioration of persistent HIV infection. A therapeutically effective dose further refers to that amount of the ligand/inhibitor sufficient to result in the reduction, prevention or inhibition of HIV trafficking and progression. For example, when in vivo administration of an ligand/inhibitor is employed, normal dosage amounts may vary from about 10 ng/kg to up to 100 mg/kg of subject body weight or more per dosage or per day, preferably about 1 pg/kg to 50 mg/kg, optionally about 100 μg/kg to 20 mg/kg, 500 μg/kg to 10 mg/kg, or 1 mg/kg to 10 mg/kg, depending upon the route of administration.

The terms “a” or “an” refers to one or more. For example, “an expression cassette” is understood to represent one or more such cassettes. As such, the terms “a” (or “an”), “one or more,” and “at least one” are used interchangeably herein.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or process steps. The words “consist”, “consisting”, and its variants, are to be interpreted exclusively, rather than inclusively, i.e., to exclude components or steps not specifically recited.

### Therapeutic Methods

In one embodiment a method of reducing or inhibiting migration of HIV-infected CD4+ T cells from blood to uninfected tissues comprising contacting the infected cells with a ligand that prevents or inhibits the interaction between a T cell-surface fucosylated glycan and its glycan-binding protein or a molecule that metabolically inhibits glycosylation or fucosylation in said cell. In one aspect of this method the HIV-infected cells are transcriptionally activated. In another embodiment of this method, the ligand is an anti-glycan antibody or binding fragment thereof. In another embodiment, the ligand is an anti-glycan binding protein or a binding fragment thereof. In still another embodiment, the ligand is an antibody of binding fragment thereof that binds to a protein in the fucose pathway and inhibits fucosylation in the cell. In another embodiment, the ligand is a small chemical compound or molecule that binds to the glycan. In yet another embodiment, the ligand is a small chemical compound or molecule that binds to the glycan binding protein. In still another embodiment, the ligand is a small chemical compound or molecule that inhibits fucosylation in the cell.

In the practice of this method in which the T cell surface glycan is a fucosylated Sialyl Lewis X (SLe^{x}) glycan, the ligand is an anti-SLe^{x} antibody or binding fragment thereof, or SLe^{x} small molecule inhibitor such as those identified above. Contacting the T cells (in vivo or in vitro) with the anti-SLe^{x} antibody or binding fragment or inhibitor prevents or inhibits interaction between the glycan and its Selectin binding partner.

Alternatively, in the practice of this method in which the T cell surface glycan is a fucosylated SLe^{x}, the ligand can be a Selectin inhibitor such as those described above. Contacting the T cells (in vivo or in vitro) with an anti-Selectin antibody or binding fragment or Selectin inhibitor also prevents or inhibits interaction between the glycan and its Selectin binding partner. In one embodiment of this method the Selectin inhibitor or antibody targets a single Selectin selected from P, L or E selections. In another embodiment, the Selectin ligand is a pan-Selectin inhibitor as described above.

Still another embodiment of the method uses a combination of different Selectin inhibitors. In one embodiment, the combination of Selectin inhibitors is a physical mixture of different Selectin specific inhibitors. In the event the inhibitors are monoclonal antibodies, the combined inhibitor can be a bi-functional or bi-specific antibody, or any other multi-binding construct. Alternatively, the mixture can be a physical admixture of chemical compounds. Still another embodiment of a mixture involves simply administering each Selectin inhibitor substantially simultaneously or sequentially, but as separate compositions.

Yet another embodiment of the method involves use as the ligand a metabolic inhibitor of glycosylation of which many are commercially available and described above or in the documents incorporated herein by reference. In another embodiment, the method uses a metabolic inhibitor of cell fucosylation, which can be the 2FF compound identified herein. By inhibiting the production of fucosylation and particularly a fucosylated glycan, such as SLe^{x}, on the cell surface, the amount of fucose is reduced. As described above and in the figures, this result is intended to reduce or suppress HIV persistence in the cell by reducing or inhibiting the ability of the cell to traffic the virus from the blood to surrounding as yet uninfected tissue.

In yet a further embodiment, the ligand or molecule is a bi-specific ligand or molecule, in which one specificity targets the glycan or its binding partner and the other specificity targets a cell surface protein on an HIV-1 infected cell such as CD4. Such dual specificity can provide an additional targeting mechanism by keeping the desired inhibitors in the environment of the HIV-infected cells.

In still a further embodiment, the method when used in vivo can further include a step of administering to the subject receiving the ligand antiretroviral ART therapy (ART).

Thus, another aspect of this invention is a method of reducing HIV persistence comprising administering to a subject in need thereof a therapeutic agent that interferes with the binding between SLe^{x} and Selectin, or that metabolically inhibits fucosylation or glycosylation, substantially simultaneously with ART therapy. The therapeutic agent can be administered as an SLe^{x} inhibitor, a Selectin inhibitor or a metabolic inhibitor of fucosylation. This administration can occur before the subject receives ART. In another embodiment, the administration of the therapeutic agent can occur after the subject receives ART. In still another embodiment, the administration of the therapeutic reagent can occur during the course of ART. In one embodiment, it is anticipated that the administration of the therapeutic agent will occur as a single dose. In another embodiment, the administration is in multiple separate dosages. In still another protocol, the therapeutic reagent is administered for a time sufficient to reduce fucosylation in the HIV-infected cells and thus reduce trafficking of the HIV to new uninfected tissues. Thus, one protocol involves administering the therapeutic reagent for a set period of time; stopping the administration of therapeutic agent, but continuing ART.

It is anticipated that a physician will determine appropriate timing and dosing protocols for this method taking into consideration the subject's physical condition, and level of detectable viremia.

In still another embodiment, this method can include periodic evaluations of the fucosylation levels by use of the glycomic signature and diagnostic methods also disclosed herein.

In yet another embodiment of the methods described herein is a method of stimulating cellular immunity in an HIV infected subject by altering the glycans on the cell surface of cells infected with HIV, or manipulating host cell-surface glycan-lectin interactions. This method is also accomplished by contacting the infected cells with a ligand that prevents or inhibits the interaction between a T cell-surface fucosylated glycan and its glycan-binding protein or a molecule that metabolically inhibits glycosylation or fucosylation in said cell. By administering the ligands or therapeutic agents that reduce fucosylated glycans expressed on the cells, as discussed in the alternative methods above, one may not only reduce trafficking of the HIV from infected cells to uninfected tissues but also improve cellular immunity.

With regard to all of these methods, it is anticipated that one of skill in the art could not only target the glycans, but also target the members of the glycomic signature or the transferases, such as FUT7 to reduce glycosylation. This can be done by resort to the teachings provided herein and knowledge extant in the art.

In all of the embodiments of the methods above at least one or more of the above described ligands (e.g. inhibitors) or therapeutic agents may be administered in combination with at least one or more additional anti-HIV agent. The inhibitors/ligands may function independently of the agent, or may function in coordination with the agent, e.g., by enhancing effectiveness of the additional agent or vice versa. In addition, the administration may be in conjunction with one or more other therapies for reducing toxicities. For example, at least one agent to counteract a side effect of therapy with the ligand/inhibitors or other anti-HIV therapy may be administered. At least one compound described herein may be administered before, after or simultaneous with administration of at least one agent or at least one agent to reduce a side effect of therapy. Where administration is simultaneous, the combination may be administered from a single container or two (or more) separate containers.

A therapeutically effective dosage of the ligand/inhibitors described herein can be administered by any suitable route, depending upon the nature of the ligand/inhibitor/metabolic inhibitor used. In addition to the dosages provided above, other dosages known to be effective for binding of the ligand/inhibitor/metabolic inhibitor in treatment of other conditions can be used. See, e.g., the documents incorporated by reference herein and other publicly available information on the use of the various Selectin inhibitors (both small molecule and mAB) identified herein.

Pharmaceutical compositions may be formulated for any appropriate route of administration. For example, compositions may be formulated without limitation for intravenous, parenteral, subcutaneous, intramuscular, intracranial, intraorbital, ophthalmic, intraventricular, intracapsular, intraspinal, intracisteral, intraperitoneal, intranasal, or aerosol administration. A physician having access to such information readily determine an appropriate dosage and route of administration.

### Diagnostic Methods and Reagents

Still another method that uses the glycomic signatures and HIV trafficking insights made by the inventors involves a method of detecting HIV-infected cells in a subject. In one embodiment, this diagnostic method is performed in vitro on biological samples from a human subject. Another embodiment of this method can be performed by administering the diagnostic reagent in vivo, and performing non-invasive techniques for measurement. The biological sample and/or the subject of the diagnostic test is in one embodiment an HIV-infected human subject who may be symptomatic or asymptomatic. In one embodiment, the subject may be examined for an initial diagnosis of HIV infection. In still other embodiments, the subject is an HIV infected subject, treated with ART and the purpose of the diagnostic test is to detect tissues or cells harboring HIV reservoirs indicative of persistent HIV. The diagnostic reagents and protocol may be used on a variety of such patients to determine the status of infection or to monitor the progress of treatment or to indicate the need for different treatment.

The diagnosis involves contacting a biological sample from said subject with a diagnostic reagent comprising a ligand that binds to or otherwise measurably detects a biomarker target that is indicative of increased fucosylation of the cells and/or tissue in the sample. The biomarker target is a fucosylated glycan, selected from the glycomic signature obtained by the inventors. In another embodiment the biomarker target is core fucose, or α-1-2 branched fucose or α-1-3 branched fucose or SLe^{x} or fucosylated SLe^{x}. In another embodiment, the biomarker target and/or glycomic signature includes one or more of UEAI, PSA, AAL, LCA, AOL. Still other biomarker targets include one or more of SELL, ENGase, PECAM1, MAN1C1, B4GALT4, St3GAL5, GAL3ST4, SLC2A11, SLC16A10, GPT2, ST6GALNAc1, SLC2A8, PYGL, PLD1 FUCA2, BCAT1, LGALS9, SLC16A1, FUT7, MYCBP, B4GALT1, SLC2A1, LGALS1, PFKFB3, RPS6KA1, PFKL, HK1, ICAM2, ICAM3, GAPDH, LDHA, TRIM28, BCL11B, ZBTB10, CCNT1,SERINC5 NFATC1, BACH2 AKT2 TNFRSF1OD MAPK8, ZBTB18, RELB USP18, MP3K21, IL2, TRFRSF11A, TLR3 CDK1 TP53I11, MP3K20, EIF2AK2, SLFN11, TGFBI, IFI30, BST2, APOBEC3G, TNF, CDKN1A, HLA-DRA, S100A4, IFITM3, ISG15, IFI16, CD4, IFITM2, NFKBIA (See FIG. 5).

The ligands can be antibodies, fragments and/or small molecules that bind to or attach to the biomarkers. These ligands are associated with a detectable label selected from those described above. In one embodiment, each different biomarker targeting ligand has a different label, so that the levels of such targets can be distinguished. In one embodiment, the ligand is an antibody or fragment thereof attached (covalently or by other conventional means) to a detectable label. In one embodiment, for example, the ligand is an anti-SLe^{x} antibody associated with a fluorescent label. In another embodiment, the ligand is a bi-specific ligand, which comprises a second ligand that is detectably labeled with a different detectable label and that binds a cell surface protein on an HIV-1 infected cell. Such a bi-specific ligand increases the accuracy of the targeting of HIV-infected cells by placing the ligand in the environment of HIV-infected T cells. Therefore, as another example is a bi-specific antibody, in which one part of the antibody targets SLe^{x} and the other part targets CD4 or another HIV-infected T cell surface receptor. Still other bi-specific or multi-specific ligands target multiple of the biomarkers in the glycomic signature. In still another aspect of the method the multiple labeled ligands are in a combination or mixture without being physically associated.

In one embodiment, the number of ligands used in the contacting step includes from at least one to at least 5, 10, 15, 20, 25, 30, 35, 40, 45, 50, 55, 60, 65, 70, 75, 80, 85, 90, to over 100. Once the cells in the sample are targeted with the labeled ligands, and a suitable time for the interaction between the ligand and target to occur has passed, a detecting method is performed to determine the level of expression of the targets within the samples. One of skill in the art can readily select such techniques from among many that are known. For example, where the label is fluorescent, a fluorescent imaging technique such as those described above may be performed. Increased levels of detection over that of a control sample, e.g., a healthy or non-infected, or non-viremic control can indicate that the sample contains HIV-infected cells. Such cells may be transcriptionally active as well.

Yet another embodiment of the diagnostic method described above is as an in vivo method in which the subject described above is administered the biomarker-targeting and labeled ligand(s). In instances where the ligand is administered to the subject, the labels are desirably those that can be detected by non-invasive imaging techniques, such as those described above. Thus, this method is performed by administering one or more of the labeled ligands, bi-specific or multi-specific ligands or mixtures above, and a non-invasive imaging technique is performed to detect the label(s), wherein the increase in detection of the label(s) over a control indicates the presence of HIV-infected cells. Further the use of non-invasive imaging techniques in the in vivo technique means that the location of possible HIV reservoirs can be detected, as well as their existence.

This latter in vivo method can be performed prior to HIV infection, prior to treatment for HIV infection, during treatment of HIV infection or after treatment. In another embodiment, this diagnostic method may be part of the therapeutic methods described above, or preceded or followed by application of such therapeutic methods.

### Compositions

Thus, a variety of novel compositions can be prepared for use in the methods described above. In one embodiment, a diagnostic reagent for non-invasive detection of HIV-infected cells or tissue comprises a ligand that binds to a fucose-containing cell surface molecule, associated with a fluorescent (or otherwise detectable) label, and optionally associated with a second ligand, associated with a second detectable label, that binds to cell surface marker on HIV infected T cells, such as CD4, CCRS or CXCR4.

In another embodiment, a diagnostic kit comprises one or more ligand that binds to a fucose-containing cell surface molecule, associated with a label that is detectable by non-invasive means, e.g., image; one or more second ligand that binds to a surface protein or epitope on an HIV infected T cell, a variety of different detectable labels for association with the ligands, the labels capable of being identified by non-invasive techniques.

In another embodiment, a non-invasive carbohydrate-based imaging probe (targeting fucose containing glycans such as SLe^{x}) to identify persistent HIV-infected cells in tissues. The probe can be used to reveal the dynamics of HIV infection and its reservoirs before and after HIV potential curative interventions. These carbohydrate-based imaging probes can be detected by conventional imaging techniques.

Yet another aspect provides a bi-specific composition comprising a ligand that binds to a fucose-containing cell surface molecule, associated with a ligand that binds to a surface protein or epitope expressed on an HIV infected T cell, one or more of the ligands associated with a detectable label.

Another aspect provides a composition containing a mixture of different ligands, each ligand capable of binding to a fucosylated glycan, or the fucosylated glycan binding protein, or a protein in the fucosylation pathway, e.g., a fucosylation transferase (FUT), each ligand optionally associated with a detectable label. In another embodiment, the mixture includes a mixture of metabolic inhibitors of glycosylation or fucosylation. In another embodiment, the composition includes one or more ligands that bind to a surface protein or epitope expressed on an HIV infected T cell, optionally associated with a detectable label. A pharmaceutically acceptable carrier or excipient may be included in any of these compositions.

Other compositions include diagnostic kits containing one or more of the ligands described above, a variety of detectable labels and reagents for their detection, and suitable apparatus for labeling of the ligands and collection of the samples.

Still other novel therapeutic reagents can comprise one or more of the ligands or inhibitors described above. In one embodiment, the novel ligand is a bispecific antibody comprising an antibody binding fragment of an anti-SLe′ antibody and an antibody binding fragment of cell surface marker of an HIV infected T cell, e.g., an anti-CD4 antibody. In another embodiment, the novel ligand is a bispecific antibody comprising an antibody binding fragment of an anti-Selectin antibody and an antibody binding fragment of cell surface marker of an HIV infected T cell, e.g., an anti-CD4 antibody. In another embodiment, the novel ligand is a bispecific antibody comprising an antibody binding fragment of an anti-core fucose or branched fucose antibody and an antibody binding fragment of cell surface marker of an HIV infected T cell, e.g., an anti-CD4 antibody. These reagents can be used therapeutically to treat subjects according to the therapeutic methods described herein.

Pharmaceutical compositions may be in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc.

Methods and agents well known in the art for making formulations are described, for example, in “Remington's Pharmaceutical Sciences,” Mack Publishing Company, Easton, Pa. Formulations may, for example, contain excipients, diluents such as sterile water, or saline, polyalkylene glycols such as polyethylene glycol, oils of vegetable origin, or hydrogenated napthalenes.

### EXAMPLES

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

The role of host glycosylation machinery in the persistence and immunopathogenesis of HIV has mostly been overlooked, despite the fact that aberrant glycosylation alters how the immune system perceives diseases and can induce immunomodulatory signaling. The inventors aimed to characterize the relationship between cell-surface glycomic signatures of HIV+ cells and levels of persistent HIV transcription, in vitro and in vivo.

### Example 1: Glycomic Signature on the Cd4+ T Cell Surface Associate with Persistent Hiv Transcription In Vitro

To examine if CD4+ T cell-surface glycomic features associated with HIV persistence, we started by exploiting a dual fluorescent reporter-based HIV (dfHIV2). DfHIV2 enables the differentiation and purification of primary CD4+ T cells into three categories: HIV-infected and transcriptionally inactive, HIV-infected and transcriptionally active, or uninfected (see Battivelli E et al, ref 10, incorporated by reference herein). Primary CD4+ T cells were isolated from HIV-uninfected donors, activated using αCD3/αCD28, infected with dfHIV2 by spinoculation, and sorted 4 days post-infection into the three categories above. Since HIV infection of primary CD4+ T cells requires activation, the uninfected cells after dfHIV2 infection are activated CD4+ T cells.

For more accurate control, we included a culture of non-activated CD4+ T cells from the same donors; this culture was treated identically except PBS was added instead of activating beads. We purified isolated cell-membrane proteins from each of the four populations, then used the lectin microarray to profile the cell-surface glycomic signature of the sorted cells populations. The glycomic signature of HIV+ transcriptionally inactive cells clustered distinctly from the other three populations due to the differential binding intensity of a selective set of lectins, including lectins specific for branched fucose (UEA-I) and core fucose (AOL) (FIGS. 1A and 1B). This indicates that the proteins on the surface of HIV+ transcriptionally inactive CD4+ T cells had lower levels of fucose (branched and core) than did the other T cell subsets.

### Example 2: Glycomic Signature on the Cd4+ T Cell Surface Associate with Persistent Hiv Transcription In Vivo

We validated our in vitro observations by asking whether different levels of T cell fucosylation corresponded with varying levels of persistent HIV transcription in vivo. We used fluorescently-labeled lectins and sialyl Lewis X (fucosylated antigen) antibodies to sort CD4+ T cells from HIV+ ART+ individuals into populations with low, medium, and high levels of branched or core fucose. Low-fucose sorted CD4+ T cells from HIV+ ART+ individuals exhibited lower levels of cell-associated HIV RNA when compared to cells with high cell-surface fucose (P<0.05 Wilcoxon test; 17.2 fold for core fucose and 8.2 fold for branched fucose) (see FIGS. 2A-2F). Furthermore, levels of core and branched fucosylation on CD4+ T cells significantly correlate with levels of CD4+ T cell-associated HIV RNA in HIV+ ART-suppressed individuals in vivo (see FIG. 3).

### Example 3: RNA-Seq

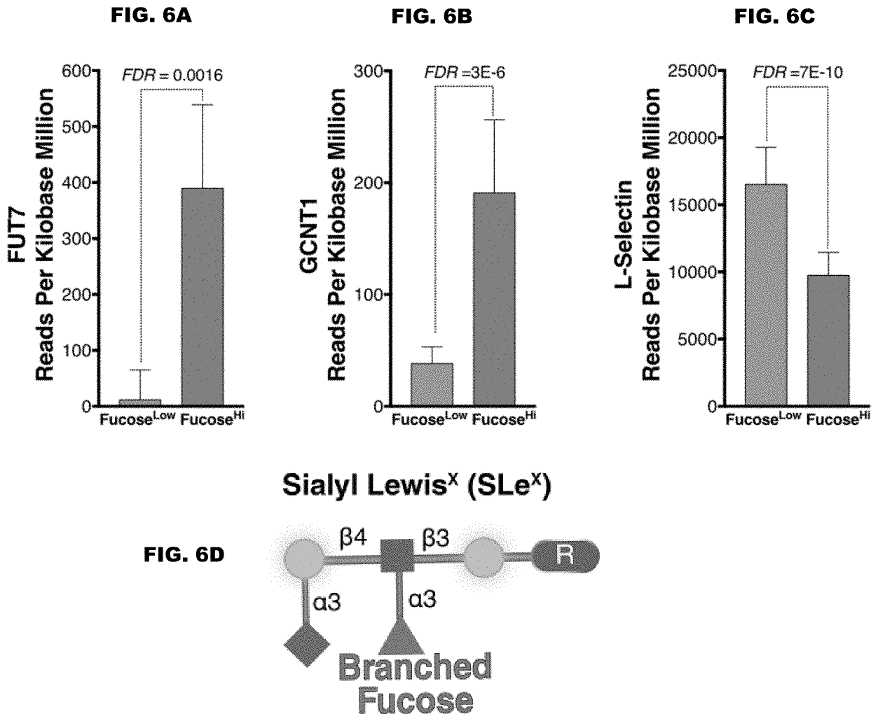

RNA-Seq was used in the sorted population to characterize the transcriptomes of high-fucose and low-fucose CD4+ T cells from HIV+ ART+ individuals. Ingenuity pathway analysis was used to evaluate the functional significance of differentially expressed genes. Principal component analysis of the transcriptomic profiles showed a clear clustering between the two groups (see, e.g., FIGS. 4A and 4B). Ingenuity pathway analysis showed that the activity of carbohydrate metabolism, glycolysis, T-cell trafficking, mTOR signaling, and ERK/MAPK signaling, being significantly elevated in high-fucose cells when compared to low-fucose cells (FDR<0.05) (see, e.g., FIG. 5). Cells with high cell-surface fucose express high levels of FUT7, an alpha-(1,3)-fucosyltransferase encodes Sialyl Lewis X (SLe^{x}), low levels of L-Selectin, and high levels of GCNT1, a gene essential for core 2 O-glycan branching (see e.g., FIGS. 6A-6C). SLe^{x} is a high fucose cell-surface antigen, binds to the cell adhesion molecules (Selectins). This binding allows leukocytes to leave the vascular tree and become recruited into tissues and sites of inflammation. In addition to the high levels of FUT7, the low levels of L-Selectin and high levels of GCNT1, suggest that these cells with high fucose are memory CD4+ T cells (a cell type known to contain the majority of HIV reservoir in blood), while the cells with low fucose are naïve CD4+ T cells.

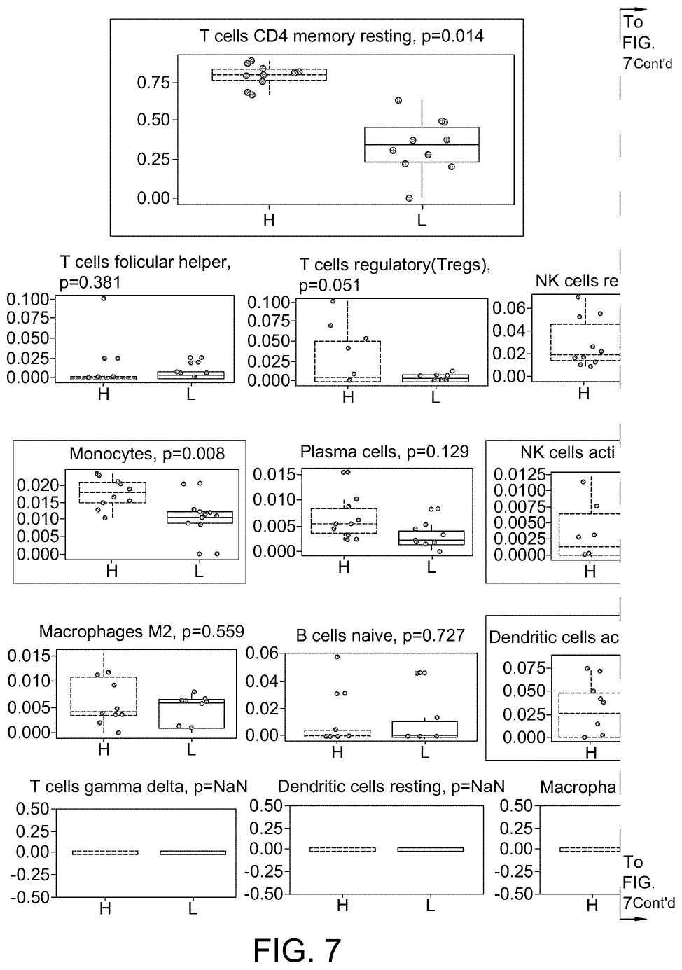

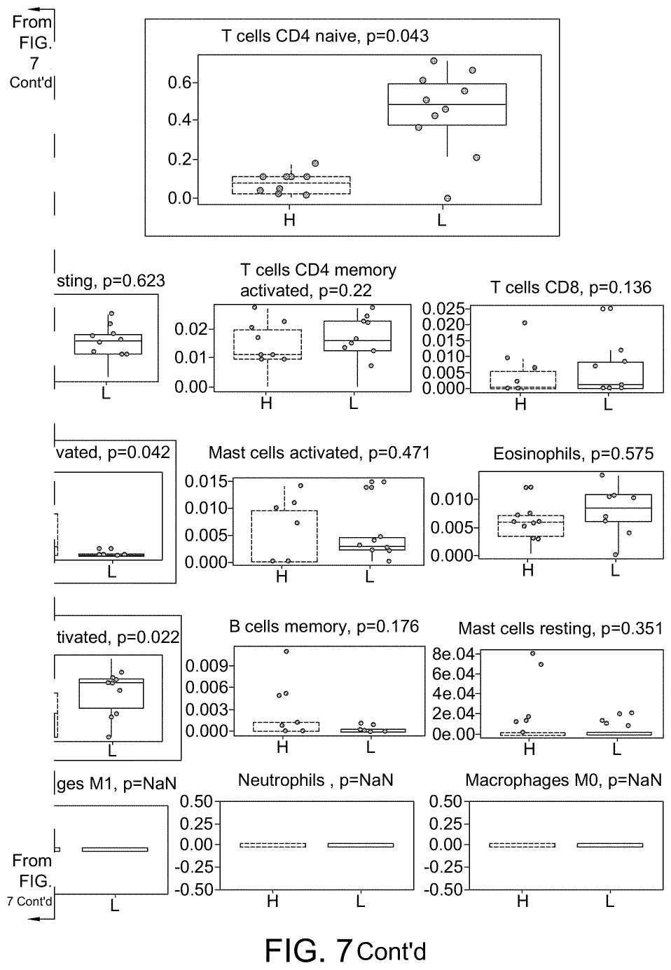

A Cybersort analysis on the RNAseq data shows that most of the fucose-high cells are likely resting memory CD4+ T cells and most of the fucose-low cells are likely naïve CD4+ T cells (see FIG. 7).

### Example 4: In Vivo Studies

The Pan-Inhibitor GMI 1070 (pan-Selectin inhibitor, currently in phase III clinical trial for treating sickle cell anemia) is used to stop the trafficking of HIV+ transcriptionally active memory CD4+T, that express high levels of SLe^{x}, from blood to tissues, allowing them to be eliminated from the body by viral cytopathic effects of immune-mediated clearance, instead of seeding HIV reservoir in tissues.

In one experiment, we investigate the impact of pan-Selectin inhibitor(s) on HIV persistence in a well-established humanized mouse model of HIV Infection. Step A: We use the physiologically-relevant bone marrow-liver-thymus (BLT) humanized mouse model of HIV infection. BLT humanized mice is the most advanced small animal model available for addressing our question.^{23,24} BLT mice allow the study of HIV infection kinetics in tissues, as human hematopoietic cells are disseminated throughout the mice.^{25,26} They also allow the study of the systemic effects of HIV infection, as the BLT human immune system recapitulates key features of HIV pathogenesis and persistence. ^{23,27-38}

We infect 30 BLT mice by intravenous injection of 3×10⁴ tissue culture infectious units HIV-1JR-CSF (NIH AIDS Reagent Program, catalog no. 394). The infection is monitored in peripheral blood for 6 weeks by measuring plasma levels of HIV RNA using one-step reverse transcriptase real-time PCR (RT-qPCR).^{36,39} ART is administered via ½″ pellets of irradiated Teklad chow 2020X containing 1500 mg emtricitabine, 1,560 mg tenofovir disoproxil fumarate, and 600 mg raltegravir per kg (Research Diets, New Brunswick, N.J.).⁴⁰ This ART regimen was chosen because of its superior efficacy in humans^{41,42} and BLT mice.^{27,40,43}

After another 6 weeks, ART-suppressed mice are divided into 6 groups (n=5 per group) and treated for 4 weeks; group 1 is treated with empty vehicle (controls), groups 2-6 are treated with different dosages of pan-Selectin-inhibitor intraperitoneally every other day for a total of 14 doses. Mice are monitored daily for signs of toxicity (weight loss, bleeding, difficulty breathing, ruffled fur, abdominal distention, reduced movement, or lethargy). During the course of the experiment, blood (100 μl) is collected every other week to 1) confirm viral suppression (by measuring HIV viral load using RT-qPCR), and 2) evaluate the longitudinal, inflammatory changes (by measuring levels of pro-inflammatory markers human sCD14 and IL-6, by ELISA. At the end of the 14 doses, blood and tissues are harvested. Single cell suspensions are prepared from bone marrow, liver, lung, lymph node, spleen, and peripheral blood.

We measure

(1) levels of CD4+ T cell-associated HIV DNA and RNA [qPCR] in blood and tissues,⁴⁷⁻⁴⁹

(2) HIV virion production, by plasma levels of HIV p24, using ultrasensitive HIV p24 assay using the Quanterix Simoa digital p24 antigen immunoassay available at Penn CFAR immunology core,

(3) plasma markers of inflammation, microbial translocation, and immune activation [sCD14, EndoCAb, I-FABP, sCD163, LPS, TNFα, IL-10, and IL-6, by Luminex], and

(4) cell-surface expression of T cell and monocyte activation and inflammation markers CD3, CD4, CD8, HLA-DR, CD38, CD69, CD25, CD14, CD16, CD163, and PD1 [FACS].

Step B: The most effective dose determined in Step A is used in Step B, 30 ART-suppressed BLT mice (infected and ART-treated as above) are divided into two groups, with one group treated for 4 weeks with 14 doses of empty vehicle (n=15) and the other treated with 14 doses of pan-Selectin inhibitor (n=15). Treatment and ART are discontinued, and plasma viremia is monitored twice a week for 6 weeks.

### Example 5: The Impact of Pan-Selectin Inhibitor(s)

The impact of pan-Selectin inhibitor(s) on SIV persistence and post-treatment-interruption viral rebound are assessed in SIV-infected ART-suppressed rhesus macaques.

The effects of pan-Selectin inhibitor(s) on HIV persistence and post-treatment-interruption viral rebound are assessed in HIV-infected ART-suppressed individuals.

Each and every patent, patent application, and publication, including the provisional application U.S. 62/770,134, and websites cited throughout specification, are incorporated herein by reference. While the invention has been described with reference to particular embodiments, it will be appreciated that modifications can be made without departing from the spirit of the invention. Such modifications are intended to fall within the scope of the appended claims.

### REFERENCES

1 Wong, J. K., et al. Recovery of replication-competent HIV despite prolonged suppression of plasma viremia. Science 278, 1291-1295 (1997).
2 Deeks, S. G. HIV infection, inflammation, immunosenescence, and aging. Annu Rev Med 62, 141-155, doi:10.1146/annurev-med-042909-093756 (2011).
3 Chun, T. W., et al. Quantification of latent tissue reservoirs and total body viral load in HIV-1 infection. Nature 387, 183-188, doi:10.1038/387183a0 (1997).
4 Finzi, D., et al. Identification of a reservoir for HIV-1 in patients on highly active antiretroviral therapy. Science 278, 1295-1300 (1997).
5 Finzi, D., et al. Latent infection of CD4+ T cells provides a mechanism for lifelong persistence of HIV-1, even in patients on effective combination therapy. Nat. Med. 5, 512-517, doi:10.1038/8394 (1999).
6 Zhang, L., et al. Quantifying residual HIV-1 replication in patients receiving combination antiretroviral therapy. N. Engl. J. Med. 340, 1605-1613, doi:10.1056/NEJM199905273402101 (1999).
7 Ramratnam, B., et al. The decay of the latent reservoir of replication-competent HIV-1 is inversely correlated with the extent of residual viral replication during prolonged anti-retroviral therapy. Nat. Med. 6, 82-85, doi:10.1038/71577 (2000).
8 Strain, M. C., et al. Heterogeneous clearance rates of long-lived lymphocytes infected with HIV: intrinsic stability predicts lifelong persistence. Proc. Natl. Acad. Sci. U.S.A 100, 4819-4824, doi:10.1073/pnas.0736332100 (2003).
9 Siliciano, J. D., et al. Long-term follow-up studies confirm the stability of the latent reservoir for HIV-1 in resting CD4+ T cells. Nat. Med. 9, 727-728, doi:10.1038/nm880 (2003).
10 Battivelli, E., et al. Distinct chromatin functional states correlate with HIV latency reactivation in infected primary CD4(+) T cells. Elife 7, doi:10.7554/eLife.34655 (2018).
11 Hirabayashi, J., et al. Lectin microarrays: concept, principle, and applications. Chemical Society reviews 42, 4443-4458, doi:10.1039/c3cs35419a (2013).
12 Tateno, H., et al. A versatile technology for cellular glycomics using lectin microarray. Methods in enzymology 478, 181-195, doi:10.1016/50076-6879(10)78008-3 (2010).
13 Tateno, H., et al. Glycome diagnosis of human induced pluripotent stem cells using lectin microarray. The Journal of biological chemistry 286, 20345-20353, doi:10.1074/jbc.M111.231274 (2011).
14 Tateno, H., et al. A novel strategy for mammalian cell surface glycome profiling using lectin microarray. Glycobiology 17, 1138-1146, doi:10.1093/glycob/cwm084 (2007).
15 Tateno, H., et al. Comparative analysis of core-fucose-binding lectins from Lens culinaris and Pisum sativum using frontal affinity chromatography. Glycobiology 19, 527-536, doi:10.1093/glycob/cwp016 (2009).
16 Nagahara, K., et al. Galectin-9 increases Tim-3+ dendritic cells and CD8+ T cells and enhances antitumor immunity via galectin-9-Tim-3 interactions. J Immunol 181, 7660-7669 (2008).
17 Uchiyama, N., et al. Optimization of evanescent-field fluorescence-assisted lectin microarray for high-sensitivity detection of monovalent oligosaccharides and glycoproteins. Proteomics 8, 3042-3050, doi:10.1002/pmic.200701114 (2008).
18 Hirabayashi, et al. Development and Applications of the Lectin Microarray. Topics in current chemistry 367, 105-124, doi:10.1007/128 2014 612 (2015).
19 Estes, J. D., et al. Defining total-body AIDS-virus burden with implications for curative strategies. Nature medicine 23, 1271-1276, doi:10.1038/nm.4411 (2017).
20 Fukazawa, Y et al. B cell follicle sanctuary permits persistent productive simian immunodeficiency virus infection in elite controllers. Nature medicine 21, 132-139, doi:10.1038/nm.3781 (2015).
21 McGary, C. S., et al. CTLA-4+PD-1-Memory CD4+ T Cells Critically Contribute to Viral Persistence in Antiretroviral Therapy-Suppressed, SIV-Infected Rhesus Macaques. Immunity 47, 776-788 e775, doi:10.1016/j.immuni.2017.09.018 (2017).
22 Perreau, M., et al. Follicular helper T cells serve as the major CD4 T cell compartment for HIV-1 infection, replication, and production. The Journal of experimental medicine 210, 143-156, doi:10.1084/jem.20121932 (2013).
23 Denton, P. W. & Garcia, J. V. Humanized mouse models of HIV infection. AIDS reviews 13, 135-148 (2011).
24 Denton, P. W. & Garcia, J. V. Mucosal HIV-1 transmission and prevention strategies in BLT humanized mice. Trends Microbiol 20, 268-274, doi:10.1016/j.tim.2012.03.007 (2012).
25 Denton, P. W., et al. IL-2 receptor gamma-chain molecule is critical for intestinal T-cell reconstitution in humanized mice. Mucosal Immunol 5, 555-566, doi:10.1038/mi.2012.31 (2012).
26 Melkus, M. W., et al. Humanized mice mount specific adaptive and innate immune responses to EBV and TSST-1. Nature medicine 12, 1316-1322, doi:10.1038/nm1431 (2006).
27 Denton, P. W., et al. Targeted cytotoxic therapy kills persisting HIV infected cells during ART. PLoS pathogens 10, e1003872, doi:10.1371/journal.ppat.1003872 (2014).
28 Council, O. D., et al. Role of Semen on Vaginal HIV-1 Transmission and Maraviroc Protection. Antimicrob Agents Chemother 59, 7847-7851, doi:10.1128/AAC.01496-15 (2015).
29 Chateau, M. L., et al. Rectal transmission of transmitted/founder HIV-1 is efficiently prevented by topical 1% tenofovir in BLT humanized mice. PloS one 8, e60024, doi:10.1371/journal.pone.0060024 (2013).
30 Zou, W., et al. Nef functions in BLT mice to enhance HIV-1 replication and deplete CD4+CD8+ thymocytes. Retrovirology 9, 44, doi:10.1186/1742-4690-9-44 (2012).
31 Kovarova, M., et al. Nanoformulations of Rilpivirine for Topical Pericoital and Systemic Coitus-Independent Administration Efficiently Prevent HIV Transmission. PLoS Pathog 11, e1005075, doi:10.1371/journal.ppat.1005075 (2015).
32 Sun, Z., et al. Intrarectal transmission, systemic infection, and CD4+ T cell depletion in humanized mice infected with HIV-1. The Journal of experimental medicine 204, 705-714, doi:10.1084/jem.20062411 (2007).
33 Brainard, D. M., et al. Induction of robust cellular and humoral virus-specific adaptive immune responses in human immunodeficiency virus-infected humanized BLT mice. Journal of virology 83, 7305-7321, doi:10.1128/JVI.02207-08 (2009).
34 Watkins, R. L., et al. In vivo analysis of Nefs role in HIV-1 replication, systemic T cell activation and CD4(+) T cell loss. Retrovirology 12, 61, doi:10.1186/s12977-015-0187-z (2015).
35 Watkins, R. L., et al. In vivo analysis of highly conserved Nef activities in HIV-1 replication and pathogenesis. Retrovirology 10, 125, doi:10.1186/1742-4690-10-125 (2013).
36 Wahl, A., et al. Human breast milk and antiretrovirals dramatically reduce oral HIV-1 transmission in BLT humanized mice. PLoS pathogens 8, e1002732, doi:10.1371/journal.ppat. 1002732 (2012).
37 Karpel, M. E., et al. BLT humanized mice as a small animal model of HIV infection. Current opinion in virology 13, 75-80, doi:10.1016/j.coviro.2015.05.002 (2015).
38 Long, B. R. & Stoddart, C. A. Alpha interferon and HIV infection cause activation of human T cells in NSG-BLT mice. Journal of virology 86, 3327-3336, doi:10.1128/JVI.06676-11 (2012).
39 Archin, N. M., et al. Valproic acid without intensified antiviral therapy has limited impact on persistent HIV infection of resting CD4+ T cells. AIDS 22, 1131-1135, doi:10.1097/QAD.0b013e3282fd6df4 (2008).
40 Tsai, P., et al. In vivo analysis of the effect of panobinostat on cell-associated HIV RNA and DNA levels and latent HIV infection. Retrovirology 13, 36, doi:10.1186/s12977-016-0268-7 (2016).
41 Rockstroh, J. K., et al. Durable efficacy and safety of raltegravir versus efavirenz when combined with tenofovir/emtricitabine in treatment-naive HIV-1-infected patients: final 5-year results from STARTMRK. J Acquir Immune Defic Syndr 63, 77-85, doi:10.1097/QAI.0b 013e31828ace69 (2013).
42 Jilek, B. L., et al. A quantitative basis for antiretroviral therapy for HIV-1 infection. Nat Med 18, 446-451, doi:10.1038/nm.2649 (2012).
43 Denton, P. W., et al. Generation of HIV latency in humanized BLT mice. J Virol 86, 630-634, doi:10.1128/JVI.06120-11 (2012).
44 Riveles, K., et al. Smoke from traditional commercial, harm reduction and research brand cigarettes impairs oviductal functioning in hamsters (Mesocricetus auratus) in vitro. Human reproduction 22, 346-355, doi:10.1093/humrep/de1380 (2007).
45 Okeley, N. M., et al. Development of orally active inhibitors of protein and cellular fucosylation. Proc Natl Acad Sci USA 110, 5404-5409, doi:10.1073/pnas.1222263110 (2013).
46 Denton, P. W., et al. Antiretroviral pre-exposure prophylaxis prevents vaginal transmission of HIV-1 in humanized BLT mice. PLoS Med 5, e16, doi:10.1371/journal.pmed.0050016 (2008).
47 Abdel-Mohsen, M., et al. Human Galectin-9 Is a Potent Mediator of HIV Transcription and Reactivation. PLoS Pathog 12, e1005677, doi:10.1371/journal.ppat.1005677 (2016).
48 Abdel-Mohsen, M., et al. Select host restriction factors are associated with HIV persistence during antiretroviral therapy. Aids 29, 411-420, doi:10.1097/QAD. 0000000000000572 (2015).
49 Wang, C., et al. Decreased HIV type 1 transcription in CCRS-Delta32 heterozygotes during suppressive antiretroviral therapy. J Infect Dis 210, 1838-1843, doi:10.1093/infdis/jiu338 (2014).
50 Fryer, H. R. et al., Increased T cell trafficking as adjunct therapy for HIV-1, PLOS Computational Biology, March 2018, 14:e1006028
51 Matsumura, Y. et al., H3K4/H3K9me3 Bivalent Chromatin Domains Targeted by Lineage-Specific DNA Methylation Pauses Adipocyte Differentiation, Molecular Cell, November 2015, 60:584-596
52 Ley, K and Kansas, G. S., Selectins in T-cell recruitment to non-lymphoid tissues and sites of inflammation, Nature Reviews Immunology, May 2004, 4:325-336
53 Hobbs, S. J. and Nolz, J. C., Regulation of T cell trafficking by enzymatic synthesis of O-glycans, Frontiers in Immunology, May 2017, 8(600)
54 Liang, W et al, Core Fucosylation of the T Cell Receptor is Required for T Cell Activation, Frontiers in Immunology, January 2018, 9:78
55 Fujii, H. et al., Core Fucosylation on T Cells, Required for Activation of T-Cell Receptor Signaling and Induction of Colitis in Mice, Is Increased in Patients with Inflammatory Bowel Disease, Gastroenterology, June 2016, 150(7):1620-1632;
56 Okada, M. et al., Blockage of Core Fucosylation Reduces Cell-Surface Expression of PD-1 and Promotes Anti-tumor Immune Responses of T Cells, Cell Reports, August 2017, 20(5):1017-1028
57 Colomb, F. et al., Breaking the Glyco-Code of HIV Persistence and Immunopathogenesis, Current HIV/AIDS Reports, February 2019, 16:151-168

1. A method of reducing or inhibiting migration of HIV-infected CD4+ T cells from blood to uninfected tissues or stimulating cellular immunity by altering the glycans on the cell surface of cells infected with HIV, or manipulating host cell-surface glycan-lectin interactions comprising contacting the infected cells with a ligand that prevents or inhibits the interaction between a T cell-surface fucosylated glycan and its glycan-binding protein or a molecule that metabolically inhibits glycosylation or fucosylation in said cell.

2. The method according to claim 1, wherein the HIV-infected CD4+ T cells are transcriptionally activated.

3. (canceled)

4. The method according to claim 1, wherein the ligand is an anti-glycan binding protein or anti-glycan antibody or a binding fragment thereof.

5. The method according to claim 4, wherein the ligand is an antibody or binding fragment thereof that binds to a protein in the fucose pathway and inhibits fucosylation in the cell.

6. The method according to claim 1, wherein the ligand is a small chemical compound or molecule that binds to the glycan or to the glycan binding protein.

7. (canceled)

8. The method according to claim 1, wherein the ligand is a small chemical compound or molecule that inhibits fucosylation in the cell.

9. The method according to claim 1, wherein the T cell surface glycan is a fucosylated Sialyl Lewis X (SLex) glycan and the glycan binding protein is a Selectin.

10. The method according to claim 9, wherein the ligand is an anti-SLex antibody or binding fragment thereof.

11. The method according to claim 9, wherein said ligand is a Selectin-specific inhibitor or a combination of different Selectin inhibitors.

12. The method according to claim 9, wherein said ligand is a pan-Selectin inhibitor.

13. (canceled)

14. The method according to claim 1, wherein the molecule is a metabolic inhibitor of glycosylation.

15. The method according to claim 1, wherein the molecule is a metabolic inhibitor of cell fucosylation.

16. The method according to claim 1, wherein the T cell surface glycan is a fucosylated Sialyl Lewis X (SLex) glycan and the glycan binding protein is a Selectin.

17. The method according to claim 1, wherein the ligand or molecule is a bi-specific ligand or molecule, which is associated with a second ligand or second molecule that binds a cell surface protein on an HIV-1 infected cell.

18. The method according to claim 17, wherein said HIV-infected cell surface protein is CD4.

19. The method according to claim 1, wherein said contacting comprises administering to a subject in need thereof the ligand or molecule and further simultaneously administering of antiretroviral ART therapy (ART) to said subject.

20. A method of reducing HIV persistence comprising administering to a subject in need thereof a therapeutic agent that binds SLe^{x}, Selectin or that metabolically inhibits fucosylation or glycosylation, substantially simultaneously with ART therapy.

21. (canceled)

22. The method according to claim 20, wherein said therapeutic agent is a Selectin ligand, a pan-Selectin inhibitor, a Selectin specific inhibitor, or a combination of said Selectin inhibitors.

23. (canceled)

24. A method of detecting HIV-infected cells in a subject comprising administering to said subject a ligand that binds to fucose-containing cell surface molecules, said ligand associated with a detectable label and performing a non-invasive imaging technique to detect said label, wherein the increase in detection of the label over a control indicates the presence of HIV-infected cells.

25-27. (canceled)

28. A diagnostic reagent for non-invasive detection of HIV-infected cells or tissue comprising a ligand that binds to a fucose-containing cell surface molecule, associated with a fluorescent label, and associated with a second ligand, associated with a second detectable label, that binds to CD4.
